(19) United States
(12) Patent Application Publication
(10) Pub. No.: US 2013/0331574 A1
(43) Pub. Date: Dec. 12, 2013

(54) Process for Preparing Piperazine

(71) Applicant: BASF SE, Ludwigshafen (DE)

(72) Inventors: Roland Bou Chedid, Mannheim (DE); Johann-Peter Melder, Bohl-lggelheim (DE); Ulrich Abel, Schifferstadt (DE); Roman Dostalek, Neuleiningen (DE); Nina Challand, Mannheim (DE); Bernd Stein, Alsbach-Hahnlein (DE); Michael Jodecke, Bobenheim-Roxheim (BE)

(21) Appl. No.: 13/910,602

(22) Filed: Jun. 5, 2013

Related U.S. Application Data
(60) Provisional application No. 61/656,053, filed on Jun. 6, 2012.

(51) Int. Cl. C07D 295/023

(52) U.S. Cl. CPC C07D 295/023
USPC 544/358

(57) ABSTRACT

Process for preparing piperazine of the formula I

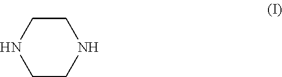

by reacting diethanolamine (DEOA) of the formula II

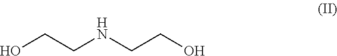

with ammonia (NH₃) in the presence of hydrogen and a supported, metal-containing catalyst,
wherein the catalytically active mass of the catalyst, prior to its reduction with hydrogen, comprises
20 to 85% by weight of oxygen-containing compounds of zirconium, calculated as ZrO₂,
1 to 30% by weight of oxygen-containing compounds of copper, calculated as CuO,
14 to 70% by weight of oxygen-containing compounds of nickel, calculated as NiO, and
0 to 5% by weight of oxygen-containing compounds of molybdenum, calculated as MoO₃,
and the reaction is carried out in the liquid phase at an absolute pressure in the range from 160 to 220 bar, a temperature in the range from 180 to 220° C., using ammonia in a molar ratio to DEOA used of from 5 to 20 and in the presence of 0.2 to 9.0% by weight of hydrogen, based on the total amount of DEOA used and ammonia.

### CROSS-REFERENCE TO RELATED APPLICATIONS

This application claims benefit (under 35 U.S.C. §119(e)) of U.S. Provisional Application 61/656,053, filed Jun. 6, 2012, which is incorporated herein by reference in its entirety.

### BACKGROUND OF THE INVENTION

### Description

The present invention relates to a process for preparing piperazine of the formula I

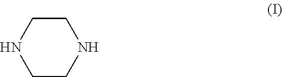

by reacting diethanolamine (DEOA) of the formula II

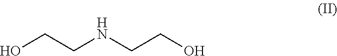

with ammonia (NH₃) in the presence of hydrogen and a supported, metal-containing catalyst.

Piperazine is used inter alia as an intermediate in the production of fuel additives (U.S. Pat. No. 3,275,554 A; DE 21 25 039 A and DE 36 11 230 A), surfactants, medicaments and crop protection compositions, hardeners for epoxy resins, catalysts for polyurethanes, intermediates for producing quaternary ammonium compounds, plasticizers, corrosion inhibitors, synthetic resins, ion exchangers, textile auxiliaries, dyes, vulcanization accelerators and/or emulsifiers.

WO 03/051508 A1 (Huntsman Petrochemical Corp.) relates to processes for the amination of alcohols using specific Cu/Ni/Zr/Sn—containing catalysts which, in a further embodiment, comprise Cr instead of Zr (see page 4, lines 10-16). The catalysts described in this WO application comprise no aluminum oxide and no cobalt.

WO 2008/006750 A1 (BASF AG) relates to certain Pb, Bi, Sn, Sb and/or In-doped, zirconium dioxide-, copper-, nickel- and cobalt-containing catalysts and their use in processes for preparing an amine by reacting a primary or secondary alcohol, aldehyde and/or ketone with hydrogen and ammonia, a primary or secondary amine. Aluminum oxide supports are not taught.

WO 2009/080507 A1 (BASF SE) describes certain Sn and Co-doped, zirconium dioxide-, copper- and nickel-containing catalysts and their use in processes for preparing an amine by reacting a primary or secondary alcohol, aldehyde and/or ketone with hydrogen and ammonia, a primary or secondary amine. Aluminum oxide supports are not taught.

WO 2009/080506 A1 (BASF SE) describes certain Pb, Bi, Sn, Mo, Sb and/or P-doped, zirconium dioxide-, nickel- and iron-containing catalysts and their use in processes for preparing an amine by reacting a primary or secondary alcohol, aldehyde and/or ketone with hydrogen and ammonia, a primary or secondary amine. Aluminum oxide supports are not taught. Preferably, the catalysts comprise no Cu and no Co.

WO 2009/080508 A1 (BASF SE) teaches certain Pb, Bi, Sn and/or Sb-doped, zirconium dioxide-, copper-, nickel-, cobalt- and iron-containing catalysts and their use in processes for preparing an amine by reacting a primary or secondary alcohol, aldehyde and/or ketone with hydrogen and ammonia, a primary or secondary amine. Aluminum oxide supports are not taught.

WO 2011/067199 A1 (BASF SE) relates to certain aluminum oxide-, copper-, nickel-, cobalt- and tin-containing catalysts and their use in processes for preparing an amine from a primary or secondary alcohol, aldehyde and/or ketone.

WO 2011/157710 A1 (BASF SE) describes the preparation of certain cyclic tertiary methylamines, where an aminoalcohol from the group 1,4-aminobutanol, 1,5-aminopentanol, aminodiglycol (ADG) or aminoethylethanolamine, is reacted with methanol at elevated temperature in the presence of a copper-containing heterogeneous catalyst in the liquid phase.

WO 2012/049101 A1 (BASF SE) relates to a process for preparing certain cyclic tertiary amines by reacting an aminoalcohol from the group 1,4-aminobutanol, 1,5-aminopentanol, aminodiglycol (ADG) or aminoethylethanolamine with a certain primary or secondary alcohol at elevated temperature in the presence of a copper-containing heterogeneous catalyst in the liquid phase.

CN 102 304 101 A (Shaoxing Xingxin Chem. Co., Ltd.) relates to the simultaneous preparation of piperazine and N-alkylpiperazines by reacting N-hydroxyethyl-1,2-ethanediamine with primary C₁₋₇-alcohols in the presence of metallic catalysts.

DE 198 59 776 A1 (BASF AG) relates to certain amination processes using catalyst moldings which comprise oxygen-containing compounds of titanium and of copper and metallic copper.

EP 382 049 A1 (BASF AG) discloses catalysts which comprise oxygen-containing zirconium, copper, cobalt and nickel compounds, and processes for the hydrogenating amination of alcohols. The preferred zirconium oxide content of these catalysts is 70 to 80% by weight (loc. cit.: page 2, last paragraph; page 3, 3rd paragraph; Examples). Although these catalysts are characterized by good activity and selectivity, they exhibit service lives which are in need of improvement. The preparation of inter alia piperazines from polybasic alcohols is mentioned on page 4, lines 49-50.

EP 696 572 A (BASF AG) relates to aminating hydrogenations using ZrO₂/CuO/NiO/MoO₃ catalysts. The preparation of inter alia piperazines from polybasic alcohols is mentioned on page 4, lines 39-40.

### BRIEF DESCRIPTION OF THE FIGURES

FIG. 1 schematically shows a particularly preferred embodiment of the integrated process.

FIG. 2 shows in a diagram form, a further particularly preferred embodiment of the integrated process.

FIG. 3 shows a diagrammatic embodiment from the prior art.

### BRIEF SUMMARY OF THE INVENTION

The object of the present invention was to improve the economic feasibility of processes to date for the preparation of piperazine of the formula I and to overcome one or more disadvantages of the prior art. The aim was to find conditions which can be established in technical terms in a simple manner and which make it possible to carry out the process with high conversion, high yield, space-time yields (STY), selectivity coupled with simultaneously high mechanical stability of the catalyst molding and low “runaway risk”.

[Space-time yields are given in “amount of product/(catalyst volume·time)” (kg/(I_{cat.}·h)) and/or “amount of product/(reactor volume·time)” (kg/(I_{reactor}·h)].

### A DETAILED DESCRIPTION OF THE INVENTION

Accordingly, a process for the preparation of piperazine of the formula I

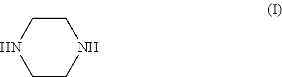

by reacting diethanolamine (DEOA) of the formula II

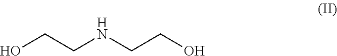

with ammonia (NH₃) in the presence of hydrogen and a supported, metal-containing catalyst has been found, wherein the catalytically active mass of the catalyst, prior to its reduction with hydrogen, comprises
20 to 85% by weight of oxygen-containing compounds of zirconium, calculated as ZrO₂,
1 to 30% by weight of oxygen-containing compounds of copper, calculated as CuO,
14 to 70% by weight of oxygen-containing compounds of nickel, calculated as NiO, and
0 to 5% by weight of oxygen-containing compounds of molybdenum, calculated as MoO₃,
and the reaction is carried out in the liquid phase at an absolute pressure in the range from 160 to 220 bar, a temperature in the range from 180 to 220° C., using ammonia in a molar ratio to DEOA used of from 5 to 20 and in the presence of 0.2 to 9.0% by weight of hydrogen, based on the total amount of DEOA used and ammonia.

The process can be carried out continuously or discontinuously. Preference is given to a continuous procedure.

In the circulating-gas procedure, the starting materials (DEOA, ammonia) are evaporated in a circulating-gas stream and passed to the reactor in gaseous form.

The starting materials (DEOA, ammonia) can also be evaporated as aqueous solutions and be passed with the circulating-gas stream to the catalyst bed.

Preferred reactors are tubular reactors. Examples of suitable reactors with circulating-gas stream can be found in Ullmann's Encyclopedia of Industrial Chemistry, 5th Ed., Vol. B 4, pages 199-238, “Fixed-Bed Reactors”.

Alternatively, the reaction takes place advantageously in a tube-bundle reactor or in a mono-stream plant.

In a mono-stream plant, the tubular reactor in which the reaction takes place can consist of a serial connection of a plurality (e.g. two or three) of individual tubular reactors. Optionally, an intermediate introduction of feed (comprising the DEOA and/or ammonia and/or H₂) and/or circulating gas and/or reactor discharge from a downstream reactor is advantageously possible here.

The circulating-gas amount is preferably in the range from 40 to 1500 m³ (at atmospheric pressure)/[m³ of catalyst (bed volume)·h], in particular in the range from 60 to 750 m³ (at atmospheric pressure)/[m³ of catalyst (bed volume)·h], further particularly preferably in the range from 100 to 400 m³ (at atmospheric pressure)/[m³ of catalyst (bed volume)·h]. (Atmospheric pressure=1 bar abs.).

The circulating gas comprises preferably at least 10, particularly 50 to 100, very particularly 80 to 100, % by volume of H₂.

In the process according to the invention, the catalysts are used preferably in the form of catalysts which consist only of catalytically active mass and optionally a shaping auxiliary (such as e.g. graphite or stearic acid), if the catalyst is used as moldings, i.e. comprise no further catalytically active accompanying substances.

In this connection, the oxidic support material zirconium dioxide (ZrO₂) is deemed as belonging to the catalytically active mass.

In the case of the zirconium dioxide, the monoclinic, tetragonal or cubic modification is preferred. Particular preference is given to the monoclinic modification.

The catalysts are used by introducing the catalytically active mass ground to powder into the reaction vessel, or by arranging the catalytically active mass after grinding, mixing with shaping auxiliaries, shaping and heat-treating as catalyst moldings—for example as tablets, beads, rings, extrudates (e.g. strands)—in the reactor.

The concentration data (in % by weight) of the components of the catalyst refer in each case—unless stated otherwise—to the catalytically active mass of the finished catalyst after its last heat treatment and before its reduction with hydrogen.

The catalytically active mass of the catalyst, after its last heat treatment and before its reduction with hydrogen, is defined as the sum of the masses of the catalytically active constituents and of the aforementioned catalyst support material and comprises essentially the following constituents:

zirconium dioxide (ZrO₂) and oxygen-containing compounds of copper and nickel and optionally molybdenum.

The sum of the aforementioned constituents of the catalytically active mass is usually 70 to 100% by weight, preferably 80 to 100% by weight, particularly preferably 90 to 100% by weight, particularly >95% by weight, very particularly >98% by weight, in particular >99% by weight, e.g. particularly preferably 100% by weight.

The catalytically active mass of the catalysts according to the invention and used in the process according to the invention can further comprise one or more elements (oxidation state 0) or inorganic or organic compounds thereof selected from groups I A to VI A and I B to VII B and VIII of the Periodic Table of the Elements.

Examples of such elements and their compounds are:

transition metals, such as Mn and MnO₂, Mo and MoO₃, W and tungsten oxides, Ta and tantalum oxides, Nb and niobium oxides or niobium oxalate, V and vanadium oxides and vanadyl pyrophosphate; lanthanides, such as Ce and CeO₂ or Pr and Pr2O₃; alkaline earth metal oxides, such as SrO; alkaline earth metal carbonates, such as MgCO₃, CaCO₃ and BaCO₃; alkali metal oxides, such as Na₂O, K₂O; alkali metal carbonates, such as Li2CO₃, Na₂CO₃ and K₂CO₃; boron oxide (B₂O₃).

Preferably, the catalytically active mass of the catalyst used in the process according to the invention comprises no rhenium, no ruthenium, no iron and/or no zinc, in each case neither in metallic (oxidation state=0) nor in an ionic (oxidation state 0), in particular oxidized, form.

Preferably, the catalytically active mass of the catalyst used in the process according to the invention comprises no silver, in each case neither in metallic (oxidation state=0) nor in an ionic (oxidation state≠0), in particular oxidized, form.

Preferably, the catalytically active mass of the catalyst used in the process according to the invention comprises no cobalt, in each case neither in metallic (oxidation state=0) nor in an ionic (oxidation state≠0), in particular oxidized, form.

Preferably, the catalytically active mass of the catalyst comprises no oxygen-containing compounds of silicon and/or of chromium.

Preferably, the catalytically active mass of the catalyst comprises no oxygen-containing compounds of titanium and/or of aluminum.

In a particularly preferred embodiment, the catalytically active mass of the catalysts according to the invention and catalysts used in the process according to the invention comprises no further catalytically active component, neither in elemental (oxidation state=0) nor in ionic (oxidation state≠0) form.

In the particularly preferred embodiment, the catalytically active mass is not doped with further metals or metal compounds.

However, customary accompanying trace elements originating from the metal extraction of Cu, Ni, Mo are excluded from this.

The catalysts can be produced by known processes, e.g. by precipitation, precipitation on, impregnation.

Preferred heterogeneous catalysts comprise in their catalytically active mass, prior to reduction with hydrogen,

20 to 85% by weight, preferably 20 to 65% by weight, particularly preferably 22 to 40% by weight, of oxygen-containing compounds of zirconium, calculated as ZrO₂,

1 to 30% by weight, particularly preferably 2 to 25% by weight, of oxygen-containing compounds of copper, calculated as CuO,

14 to 70% by weight, preferably 15 to 50% by weight, particularly preferably 21 to 45% by weight, of oxygen-containing compounds of nickel, calculated as NiO, where preferably the molar ratio of nickel to copper is greater than 1, in particular greater than 1.2, very particularly 1.8 to 8.5, and

0 to 5% by weight, particularly 0.1 to 3% by weight, of oxygen-containing compounds of molybdenum, calculated as MoO₃.

Particularly preferred heterogeneous catalysts in the process according to the invention are catalysts disclosed in EP 382 049 A (BASF AG), or correspondingly preparable, the catalytically active mass of which, prior to treatment with hydrogen, comprises

20 to 85% by weight, preferably 70 to 80% by weight, of ZrO₂,
1 to 30% by weight, preferably 1 to 10% by weight, of CuO,
and in each case 1 to 40% by weight, preferably 5 to 20% by weight, of NiO,
catalysts disclosed in EP 696 572 A (BASF AG), the catalytically active mass of which, prior to reduction with hydrogen, comprises 20 to 85% by weight of ZrO₂, 1 to 30% by weight of oxygen-containing compounds of copper, calculated as CuO, 30 to 70% by weight of oxygen-containing compounds of nickel, calculated as NiO, 0.1 to 5% by weight of oxygen-containing compounds of molybdenum, calculated as MoO₃, and 0 to 10% by weight of oxygen-containing compounds of aluminum and/or manganese, calculated as Al₂O₃ or MnO₂, for example the catalyst disclosed in loc. cit., page 8 having the composition 31.5% by weight of ZrO₂, 50% by weight of NiO, 17% by weight of CuO and 1.5% by weight of MoO₃.

The catalysts produced can be stored as they are. Prior to being used as catalysts in the process according to the invention, they are pre-reduced (=activation of the catalyst) by treating with hydrogen. However, they can also be used without pre-reduction, in which case they are then reduced (=activated) under the conditions of the process according to the invention by the hydrogen present in the reactor.

For the purposes of activation, the catalyst is exposed to a hydrogen-containing atmosphere or a hydrogen atmosphere at a temperature in the range from 100 to 500° C., particularly 150 to 400° C., very particularly 180 to 300° C., over a period of at least 25 min., particularly at least 60 Min. The activation period of the catalyst can be up to 1 h, particularly up to 12 h, in particular up to 24 h.

During this activation, at least some of the oxygen-containing metal compounds present in the catalysts are reduced to give the corresponding metals, meaning that these are present together with the different types of oxygen compounds in the active form of the catalyst.

The process according to the invention is preferably carried out continuously, the catalyst preferably being arranged as a fixed bed in the reactor. In this connection, flow through the fixed catalyst bed from above and also from below is possible.

The ammonia is used in a 5- to 20-fold molar amount, preferably 6- to 18-fold molar amount, further preferably 7- to 17-fold molar amount, particularly 9- to 16-fold molar amount, in particular in a 10- to 15-fold molar amount, e.g. 12- to 14-fold molar amount, in each case based on the DEOA used.

The ammonia can be used as aqueous solution, particularly as 30 to 90% strength by weight aqueous solution. It is preferably used without further solvent (compressed gas, purity particularly 95 to 100% strength by weight).

The starting material DEOA is preferably used as aqueous solution, particularly as 75 to 95% strength by weight aqueous solution, e.g. 80% strength by weight aqueous solution.

Preferably, an offgas amount of from 1 to 800 cubic meters (stp)/(cubic meters of catalyst·h), in particular 2 to 200 cubic meters (stp)/(m³ of catalyst·h) is processed. [Cubic meters (stp)=volume converted to standard temperature and pressure conditions (20° C., 1 bar abs.)]. Catalyst volume data always refers to the bulk volume.

The amination of the primary alcohol groups of the starting material DEOA is carried out in the liquid phase. Preferably, the fixed bed process is in the liquid phase.

When working in the liquid phase, the starting materials (DEOA, ammonia) are passed, preferably simultaneously, in liquid phase at pressures of from 16.0 to 22.0 MPa (160 to 220 bar), preferably 17.0 to 22.0 MPa, further preferably 18.0 to 21.0 MPa, further preferably 19.0 to 20.0 MPa, and temperatures of from 180 to 220° C., particularly 185 to 215° C., preferably 190 to 210° C., in particular 190 to 205° C., including hydrogen over the catalyst, which is usually located in a fixed-bed reactor heated preferably from the outside. Here, both a trickle mode and also a liquid-phase mode is possible. The catalyst hourly space velocity is generally in the range from 0.3 to 0.8, preferably 0.4 to 0.7, particularly preferably 0.5 to 0.6 kg, of DEOA per liter of catalyst (bed volume) and per hour (DEOA calculated as 100% strength). Optionally, the starting materials can be diluted with a suitable solvent, such as water, tetrahydrofuran, dioxane, N-methylpyrrolidone or ethylene glycol dimethyl ether. It is expedient to heat the reactants even before they are introduced into the reaction vessel, preferably to the reaction temperature.

The reaction is carried out in the presence of 0.2 to 9.0% by weight of hydrogen, particularly in the presence of 0.25 to 7.0% by weight of hydrogen, further particularly in the presence of 0.3 to 6.0% by weight of hydrogen, very particularly in the presence of 0.4 to 5.0% by weight of hydrogen, in each case based on the total amount of DEOA used and ammonia.

The pressure in the reaction vessel which arises from the sum of the partial pressures of the ammonia, of the DEOA and of the reaction products formed, and also optionally of the co-used solvent at the stated temperatures, is expediently increased to the desired reaction pressure by injecting hydrogen.

In the case of continuous operation in the liquid phase, the excess ammonia can be circulated together with the hydrogen.

If the catalyst is arranged as a fixed bed, it can be advantageous for the selectivity of the reaction to mix the catalyst moldings in the reactor with inert packings, to “dilute” them so to speak. The fraction of the packings in such catalyst preparations can be 20 to 80, particularly 30 to 60 and in particular 40 to 50, parts by volume.

The water of reaction formed in the course of the reaction (in each case one mole per mole of reacted alcohol group) generally does not have a disruptive effect on the degree of conversion, the rate of reaction, the selectivity and the service life of the catalyst and is therefore expediently only removed upon work-up of the reaction mixture, e.g. by distillation.

After the reaction discharge has expediently been decompressed, the excess hydrogen and the optionally present excess aminating agents are removed therefrom and the crude reaction product obtained is purified, e.g. by means of fractional rectification. Suitable work-up methods are described e.g. in EP 1 312 600 A and EP 1 312 599 A (both BASF AG). The excess primary amine and the hydrogen are advantageously returned again to the reaction zone. The same applies for any incompletely reacted DEOA.

A work-up of the product of the reaction is preferably as follows:

From the reaction product of the reaction, by means of distillation,

(i) firstly unreacted ammonia is separated off overhead,
(ii) water is separated off overhead,
(iii) optionally present by-products with a lower boiling point than that of the process product I (low boilers) are separated off overhead,
(iv) the process product piperazine (I) is separated off overhead, with optionally present by-products with a higher boiling point than that of the process product I (high boilers) and optionally present unreacted DEOA (II) remaining in the bottom.

During the reaction of the process according to the invention, the aminoethylethanolamine (AEEA) of the formula III

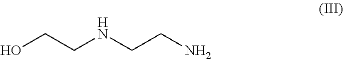

can be formed as by-product:

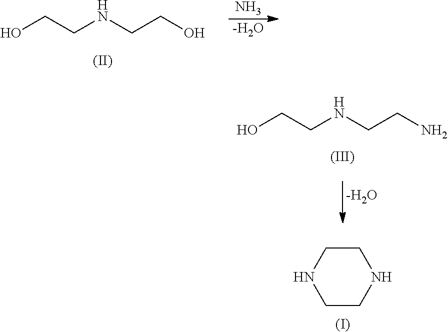

Therefore, in particular by means of distillation,

(v) from the bottom of step iv, optionally present unreacted DEOA (II) and/or optionally present aminoethylethanolamine as by-product with the formula III are separated off overhead and returned to the reaction.

Ammonia separated off in step i and having a purity of from 90 to 99.9% by weight, particularly 95 to 99.9% by weight, is preferably returned to the reaction, in which case some of the separated-off ammonia, particularly 1 to 30% by weight of the separated-off ammonia, further particularly 2 to 20% by weight of the separated-off ammonia, can be removed.

In one particular embodiment, the invention relates to an integrated, multistage process for preparing piperazine, 1,2-ethylenediamine (EDA), diethylenetriamine (N-(2-aminoethyl)-1,2-ethylenediamine, DETA) and N-(2-aminoethyl)ethanolamine (AEEA), where
(reaction stage 1=R1) in a first reaction stage ethylene oxide (EO) is reacted continuously with ammonia to give a product comprising monoethanolamine (MEOA), diethanolamine (DEOA) and triethanolamine (TEOA),
(distillation stage 1=D1) the ethanolamines MEOA, DEOA and TEOA are separated by distillation,
(reaction stage 2=R2) MEOA separated off in D1, completely or partly, preferably completely, is continuously reacted with ammonia in a second reaction stage in the presence of an amination catalyst and
(reaction stage 3=R3) DEOA separated off in D1, completely or partly, preferably completely, is reacted in a third reaction stage with ammonia by the process as described above.

Preferably, in the first reaction stage, ethylene oxide (EO) is reacted with ammonia in the presence of water as catalyst.

In particular, water and/or ammonia produced in distillation stage 1 (D1) is returned to the first reaction stage (R1).

The aminating catalyst used in the second reaction stage (R2) is preferably a Cu-containing heterogeneous catalyst, further preferably a Cu- and Ni-containing heterogeneous catalyst, particularly a Cu- and Ni- and Co-containing heterogeneous catalyst, very particularly the Cu/Ni/Co/Al₂O₃ catalyst disclosed in DE 19 53 263 A (BASF AG).

Furthermore, in an alternative embodiment, in the second reaction stage (R2), preference is given to using a Cu- and Ln-containing heterogeneous catalyst, particularly the Cu/Ln/Al₂O₃ catalyst taught in WO 2010/115759 A (BASF SE).

In reaction stage 3, particular preference is given to a procedure in which the DEOA is converted to at least 95%, particularly to 98 to 100%.

Preferably, ammonia present is separated off from the reaction product of reaction stage 2 by distillation (distillation stage 2=D2). Separated-off ammonia is advantageously returned to reaction stage 2.

Further preferably, ammonia present is separated off from the reaction product of reaction stage 3 (distillation stage 3=D3) by distillation. Separated-off ammonia is advantageously returned to reaction stage 3.

The two reaction products remaining after separating off the ammonia are preferably combined, and piperazine, EDA, DETA and AEEA and optionally present MEOA are separated off from the combined product (distillation stage 4=D4) by distillation.

In distillation stage 4 (D4), optionally present MEOA is advantageously returned to the second reaction stage (R2).

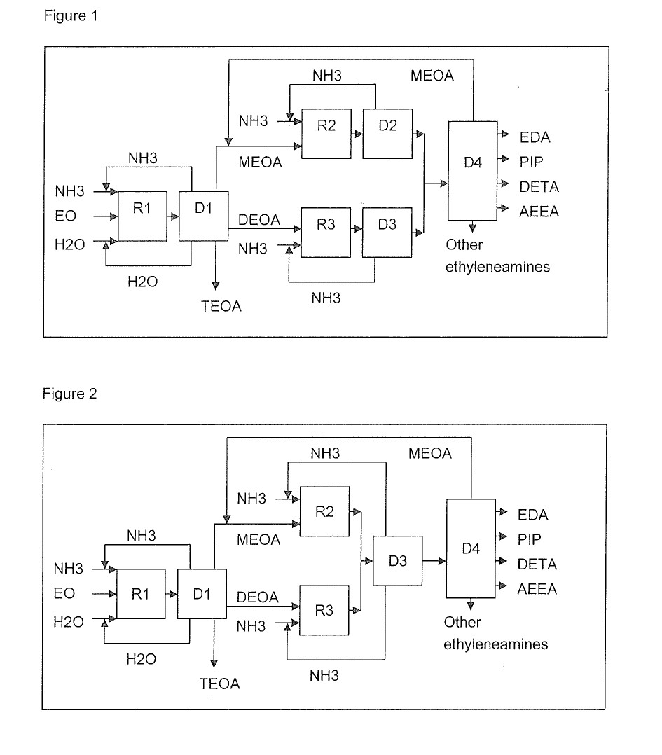

FIG. 1, accordingly, schematically shows a particularly preferred embodiment of the integrated process.

Alternatively, it is preferred to combine the reaction products from the two reaction stages R2 and R3, to separate off ammonia present from the combined product (distillation stage 3=D3) by distillation and then to separate off piperazine, EDA, DETA and AEEA and optionally present MEOA by distillation (distillation stage 4=D4).

Ammonia separated off in distillation stage 3 is advantageously returned to reaction stage 2 and/or 3.

MEOA optionally produced in distillation stage 4 (D4) is advantageously returned to the second reaction stage (R2).

FIG. 2 accordingly shows, in diagram form, a further particularly preferred embodiment of the integrated process.

All pressure data refer to the absolute pressure.

All ppm data refer to the mass.

### EXAMPLES

### 1. Preparation of Catalyst A

Catalyst A, a Cu/Ni/Mo/ZrO₂ catalyst, as disclosed in EP 696 572 A1 (BASF AG), was produced by precipitation, filtration, heat treatment and tabletting (6×3 mm tablets).

The catalyst had the following composition prior to its treatment (activation) with hydrogen:

50% by weight of NiO, 17% by weight of CuO and 1.5% by weight of MoO₃ on ZrO₂ (31.5% by weight).

### 2. Reaction of DEOA with Ammonia in a Continuously Operated Tubular Reactor

A heated tubular reactor with an internal diameter of 14 mm, a centrally installed thermocouple and a total volume of 1000 ml was filled in the lower section with a bed of glass beads (250 ml), on top of this 500 ml of the reduced catalyst A and finally the remainder was filled again with glass beads. Prior to the reaction, the catalyst was activated under atmospheric pressure for 24 hours at max. 280° C. under hydrogen (25 I(stp)/h)(I(stp)=liters at standard temperature and pressure=volume converted to standard temperature and pressure conditions (20° C., 1 bar abs.)). A certain amount of DEOA (80% strength aqueous), ammonia and hydrogen, as stated in Table I below, were metered through the reactor from bottom to top. The reactor was held at a temperature of ca. 185 to 200° C. and a total pressure of 200 bar. The reaction temperature was selected such that a DEOA conversion of >90% was reached. The mixture leaving the reactor was cooled and decompressed to atmospheric pressure. At various times, samples were taken from the reaction mixture and analyzed by means of gas chromatography. For this, an “RTX-5 amine” GC column 30 m in length was used, with a temperature program: 70° C./5 min., heat to 280° C. at a rate of 5° C./min., at 280° C./10 minutes.

The results of the experiments can be found in Table I below.

TABLE 1

| | | % by | | | | | | | | | | | | |
|---|---|---|---|---|---|---|---|---|---|---|---|---|---|---|
| | | wt. of | | | | | | | | | | | | |
| | | H₂ | HSV | | | | | | | | | | | |
| | | Based | kg/ | | | | | | | | | | | |
| | | on | (l · h) | MR | PIP | DEOA | EDA | DETA | AEEA | AEPIP | Amix | H₂O | | |
| | Temp. | DEOA + | 80% | NH₃/ | % by | % by | % by | % by | % by | % by | % by | % by | PIP sel. | EA Sel. |
| Example 2 | ° C. | NH₃ | strength | DEOA | wt. | wt. | wt. | wt. | wt. | wt. | wt. | wt. | mol % | mol % |
| A | 190 | 0.7 | 0.6 | 14 | 33.2 | 2.0 | 4.0 | 1.2 | 1.0 | 6.3 | 8.2 | 43.7 | 62 | 98 |
| B | 190 | 0.8 | 0.6 | 17 | 34.5 | 1.4 | 4.5 | 1.2 | 0.8 | 8.1 | 7.8 | 41.1 | 61 | 98 |
| C | 190 | 1.2 | 0.6 | 17 | 33.6 | 2.4 | 4.2 | 1.2 | 1.3 | 8.0 | 9.3 | 39.5 | 58 | 97 |
| D | 196 | 5.5 | 0.6 | 14 | 28.8 | 2.0 | 2.5 | 0.7 | 2 | 9 | 16.4 | 37.2 | 49 | 100 |
| E | 196 | 0.4 | 0.6 | 14 | 36.7 | 1.0 | 5.1 | 1.5 | 2.5 | 6.7 | 9.4 | 36.3 | 62 | 100 |
| F | 193 | 1.2 | 0.6 | 7 | 32.2 | 1.0 | 3.3 | 1.3 | 4.2 | 7.5 | 8.0 | 41.2 | 59 | 100 |

Pressure: 200 bar
Temp.: temperature in the reactor
HSV: catalyst hourly space velocity [kg of DEOA/(liter_{cat.} · h)]
MR: molar ratio of NH₃ to DEOA in the feed
Sel.: selectivity.
PIP sel. = piperazine selectivity;
EA sel. = selectivity of all ethyleneamines.
PIP: piperazine
AEPIP: N-(2-aminoethyl)piperazine
Amix: ethyleneamines with a higher boiling point than AEPIP

The work-up can preferably take place by means of the following five steps:

1) Separating off unreacted ammonia and returning it to the reactor Optional removal of some of the ammonia from the top of the column.
2) Separating off water
3) Separating off low-boiling secondary components
4) Pure distillation of the piperazine (I) overhead while separating off high-boiling secondary components via the bottom.
5) Optionally returning some of the high-boiling secondary components, in particular diethanolamine, N-(2-aminoethyl)ethanolamine (AEEA), N-(2-aminoethyl)ethane-1,2-diamine (diethyllenetriamine, DETA) to the reaction.

### 3. Preparation of piperazine, 1,2-ethylenediamine (EDA), diethylenetriamine (DETA) and N-(2-aminoethyl)ethanolamine (AEEA) (According to FIG. 1).

The reaction of EO with NH₃, homogeneously catalyzed with water, was carried out continuously at an NH₃:EO molar ratio (MR) of 10 (reaction stage 1).

In the process, 100 mol/h of EO produced 46.5 mol/h of MEOA, 18.7 mol/h of DEOA and 5.4 mol/h of TEOA (weight ratio: 62:29:9=MEOA:DEOA TEOA).

The ethanolamines were separated off by distillation (distillation stage 1).

The complete amount of the MEOA was reacted in reaction stage 2 in a reactor in the presence of the Cu/Ni/Co/Al₂O₃ catalyst according to DE 19 53 263 A (BASF AG), therein Example 1 on page 5, with NH₃ (molar ratio of NH₃:MEOA=8:1) in the presence of 0.5% by weight of hydrogen based on the total amount of NH₃ and MEOA at 190° C. and a catalyst hourly space velocity of 0.6 kg of MEOA/(I_{cat.}·h) to give ethyleneamines (in particular PIP, EDA, DETA, AEEA). The excess NH₃ was separated off in distillation stage D2 and returned to R2.

The complete amount of the DEOA was reacted in reaction stage 3 in a reactor in the presence of the Cu/Ni/Mo/ZrO₂ catalyst A (see above) with NH₃ as in Example 2E (Table I) to give ethyleneamines (in particular PIP, EDA, DETA, AEEA). The excess NH₃ was separated off in distillation stage D3 and returned to R3.

The products from distillation stages 2 and 3 were brought together and the ethyleneamines were separated off by distillation (distillation stage 4). Unreacted MEOA was returned to R2. Formed in total in this process by the reaction of the total amount of the ethanolamines MEOA and DEOA which were formed in reaction stage 1 (see above, 46.5 mol/h of MEOA and 18.7 mol/h of DEOA) and were separated off in distillation stage 1:
From 46.5 mol/h of MEOA: 28.83 mol/h of EDA, 3.26 mol/h of PIP, 2.56 mol/h of DETA and 2.33 mol/h of AEEA.
From 18.7 mol/h of DEOA: 11.7 mol/h of PIP, 2.3 mol/h of EDA, 0.4 mol/h of DETA, 0.6 mol/h of AEEA.
In total from 100 mol/h of EO: 31.1 mol/h of EDA, 14.9 mol/h of PIP, 3.0 mol/h of DETA, 3.0 mol/h of AEEA and 5.4 mol/h of TEOA.

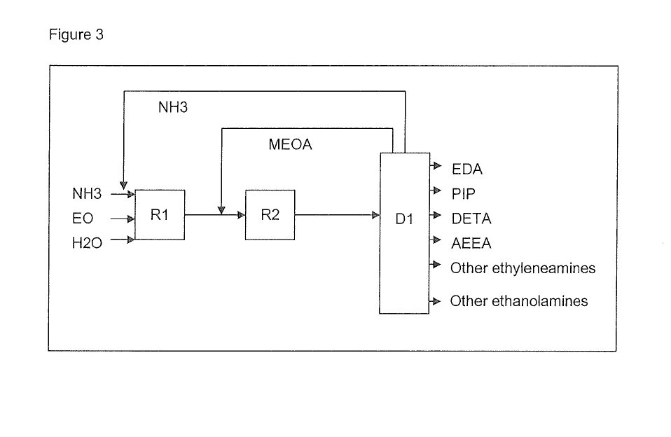

The piperazine yield based on EO was 14.9 mol % and is higher compared to the prior art (cf. also FIG. 3 for a diagrammatic embodiment from the prior art):

3.6 mol % PIP yield in EP 75940 B2, therein example on pages 10-11, 220 mol/h of EO (page 10, column 18, line 38) gives 8 mol/h of piperazine (page 11, column 20, line 34), and mol % PIP yield in WO 06/114417 A2, therein Example 2 on page 9, lines 30-40, 61 g/h (1.39 mol/h) of EO gives 6 g/h (0.07 mol/h) of piperazine.

1-32. (canceled)

33. A process for preparing piperazine of the formula I 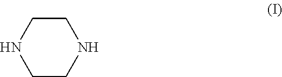
by reacting diethanolamine (DEOA) of the formula II 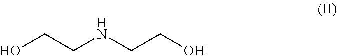
with ammonia (NH₃) in the presence of hydrogen and a supported, metal-containing catalyst,
wherein the catalytically active mass of the catalyst, prior to its reduction with hydrogen, comprises
20 to 85% by weight of oxygen-containing compounds of zirconium, calculated as ZrO₂,
1 to 30% by weight of oxygen-containing compounds of copper, calculated as CuO,
14 to 70% by weight of oxygen-containing compounds of nickel, calculated as NiO, and
0 to 5% by weight of oxygen-containing compounds of molybdenum, calculated as MoO₃,
wherein the reaction is carried out in the liquid phase at an absolute pressure in the range from 160 to 220 bar, a temperature in the range from 180 to 220° C.,
wherein the molar ratio of ammonia to DEOA is from 5 to 20, and
wherein hydrogen is present at from 0.2 to 9.0% by weight based on the total weight of DEOA and ammonia.

34. The process according to claim 33, wherein the catalytically active mass of the catalyst, prior to its reduction with hydrogen, comprises 20 to 65% by weight of oxygen-containing compounds of zirconium, calculated as ZrO₂.

35. The process according to claim 33, wherein the catalytically active mass of the catalyst, prior to its reduction with hydrogen, comprises 2 to 25% by weight of oxygen-containing compounds of copper, calculated as CuO.

36. The process according to claim 33, wherein the catalytically active mass of the catalyst, prior to its reduction with hydrogen, comprises 15 to 50% by weight of oxygen-containing compounds of nickel, calculated as NiO.

37. The process according to claim 33, wherein the catalytically active mass of the catalyst, prior to its reduction with hydrogen, comprises 0.1 to 3% by weight of oxygen-containing compounds of molybdenum, calculated as MoO₃.

38. The process according to claim 33, wherein the molar ratio of nickel to copper is greater than 1.

39. The process according to claim 33, wherein no rhenium and/or ruthenium is present in the catalytically active mass of the catalyst.

40. The process according to claim 33, wherein no iron and/or zinc is present in the catalytically active mass of the catalyst.

41. The process according to claim 33, wherein no cobalt is present in the catalytically active mass of the catalyst.

42. The process according to claim 33, wherein no oxygen-containing compounds of silicon and/or of aluminum and/or of titanium are present in the catalytically active mass of the catalyst.

43. The process according to claim 33, wherein the reaction is carried out at a temperature in the range from 185 to 215° C.

44. The process according to claim 33, wherein the reaction is carried out at an absolute pressure in the range from 170 to 210 bar.

45. The process according to claim 33, wherein the ammonia is used in a 6- to 18-fold molar amount, based on the DEOA used.

46. The process according to claim 33, wherein the reaction is carried out in the presence of from 0.25 to 7.0% by weight of hydrogen, based on the total amount of DEOA used and ammonia.

47. The process according to claim 33, wherein the catalyst is arranged as a fixed bed in the reactor.

48. The process according to claim 33, wherein the process is carried out continuously.

49. The process according to claim 48, wherein the reaction takes place in a tubular reactor.

50. The process according to claim 48, wherein the reaction takes place in a circulating-gas mode.

51. The process according to claim 33, wherein the DEOA is used as aqueous solution.

52. The process according to claim 33, wherein the ammonia is used as aqueous solution.

53. The process according to claim 33, wherein the reaction is carried out at a catalyst hourly space velocity in the range from 0.3 to 0.8 kg of DEOA/(I_{cat.}·h).

54. The process according to claim 33, further comprising distilling a reaction product of the reaction by the steps comprised of
(i) separating off overhead unreacted ammonia,
(ii) separating water off overhead,
(iii) optionally separating present by-products with a lower boiling point than that of the process product I off overhead, and
(iv) separating the piperazine of the formula I off overhead, wherein optionally present by-products with a higher boiling point than that of the piperazine of the formula I and optionally present unreacted DEOA (II) remain in the bottom.

55. The process according to claim 54, further comprising
(v) separating off overhead and returning to the reaction, by distillation, optionally present unreacted DEOA (II) and/or optionally present aminoethylethanolamine (AEEA) as by-product with the formula III 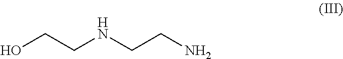
from the bottom of step iv.

56. The process according to claim 54, wherein ammonia separated off in step (i) and having a purity of from 90 to 99.9% by weight is returned to the reaction.

57. An integrated, multistage process for preparing piperazine, 1,2-ethylenediamine (EDA), diethylenetriamine (DETA) and N-(2-aminoethyl)ethanolamine (AEEA), comprising
(i) reacting ethylene oxide (EO) continuously with ammonia in a first reaction stage (R1) to give a product comprising monoethanolamine (MEOA), diethanolamine (DEOA) and triethanolamine (TEOA),
(ii) separating by distillation the MEOA, the DEOA and the TEOA in a first distillation stage (D1),
(iii) continuously reacting the MEOA separated off in D1 with ammonia in a second reaction stage (R2) in the presence of an amination catalyst, and
(iv) reacting the DEOA separated off in D1 with ammonia in a third reaction stage (R3) with ammonia by the process according to claim 33.

58. The process according to claim 57, wherein, in the first reaction stage, ethylene oxide (EO) is reacted with ammonia in the presence of water as catalyst.

59. The process according to claim 57, wherein the water and/or the ammonia produced in the first distillation stage (D1) is returned to the first reaction stage (R1).

60. The process according to claim 57, further comprising
(v) separating ammonia from the reaction product of the second reaction stage (R2) by distillation in a second distillation stage (D2).

61. The process according to claim 57, further comprising
(v) separating ammonia from the reaction product of the third reaction stage (D3) by distillation in a third distillation stage (D3).

62. The process according to claim 60, further comprising
(vi) combining the reaction product of the second reaction stage (R2) and the reaction product of the third reaction stage (R3) to form a combined product, and
(vii) separating off by distillation piperazine, EDA, DETA and AEEA and optionally present MEOA in a fourth distillation stage (D4).

63. The process according to claim 57, further comprising
(vi) combining the reaction product of the second reaction stage (R2) and the reaction product of the third reaction stage (R3) to form a combined product,
(vii) separating off by distillation ammonia in a third distillation stage (D3), and
(viii) separating off by distillation piperazine, EDA, DETA and AEEA and optionally present MEOA in a fourth distillation stage (D4).

64. The process according to claim 62, further comprising
(viii) returning the MEOA in the fourth distillation stage (D4) to the second reaction stage (R2), wherein MEOA is present in the fourth distillation stage (D4).
